(19) United States
(12) Patent Application Publication
(10) Pub. No.: US 2026/0363190 A1
(43) Pub. Date: Nov. 19, 2026

(54) Lepidium plant Named 'DANLEPGRZR'

(71) Applicant: Danziger "DAN" Flower Farm, Beit Dagan (IL)

(72) Inventor: Gavriel Danziger, Beit Dagan (IL)

(73) Assignee: Danziger "DAN" Flower Farm, Beit Dagan (IL)

(21) Appl. No.: 19/211,682

(22) Filed: May 19, 2025

(51) Int. Cl. A01H 6/20

(52) U.S. Cl. CPC A01H 6/20

(57) ABSTRACT

A new and distinct Lepidium virginicum plant named ‘DLEPVERGI’ characterized by high volume production of long flowering stems. Flowering stems are strong and sturdy. Plants require vernalization to flower. The harvested cut flower stems are well suited for commercial use as bouquet and flower arrangement filler. Plants have commercial use for cut flower production.

### FIELD OF THE INVENTION

The present invention relates to a new, distinct and stable variety ofLepidium virginicum, hereinafter referred to as ‘DANLEPGRZR’. The present invention relates to seeds which are the Lepidium virginicum ‘DANLEPGRZR’, as well as, plants and plant parts produced by these seeds which have all of the morphological and physiological characteristics of the Lepidium virginicum ‘DANLEPGRZR’. The present invention also relates to methods for producing these seeds and plants of the Lepidium virginicum ‘DANLEPGRZR’. Furthermore, the present invention relates to a method of producing progeny Lepidium plants by crossing Lepidium ‘DANLEPGRZR’, as either the female or seed or male or pollen parent, with another Lepidium plant and selecting progeny.

### BACKGROUND OF THE INVENTION

The present invention relates to a new, distinct and stable variety of Lepidium virginicum, and hereinafter referred to by the variety denomination ‘DANLEPGRZR’. The new Lepidium ‘DANLEPGRZR’ originated from a self-crossing made in a controlled breeding program by the inventor in 2018, and then first flowered in 2019, in Mishmar Hashiva, Israel. The parent is the Lepidium virginicum proprietary line identified by code LEV-17-19 (unpatented). This proprietary line is the sole parent, as the resulting ‘DANLEPGRZR’ is the result of a self-crossing of this single parent variety.

Lepidium is a member of the Brassicaceae family. Lepidium virginicums is a vascular land plant, native to North America. For the most part, plants of Lepidium virginicum are annual or biennial, producing small or minute flowers in racemes.

To the inventor's best knowledge, there have not been significant commercial efforts to date to hybridize and produce new and interesting Lepidium virginicum varieties.

Over time, the inventor has trialed Lepidium and found it may be advantageously grown for ornamental horticultural uses. Typically, the plants are tolerant of hot, sunny conditions, and require little water.

Leaves of Lepidium can be sessile or stalked, normally basally occuring. Lepidium plants produce upright spikes of hermaphroditic flowers.

Asexual propagation of Lepidium can be performed by vegetative terminal cutings, however, propagation is most commonly performed by sowing seeds.

Methods for cultivation and crossing of Lepidium are not well known. Brief reference to the species can be found in: Brassicaceae, Capparaceae and Cleomaceae of North America Update, database (version 2011)Updated for ITIS by the Flora of North America Expertise Network, in connection with an update for USDA PLANTS (2007-2010), which is herein incorporated by reference.

The Lepimedium virginicum seeds and plants produced by this method are uniform with respect their morphological and physiological characteristics.

A need exists for a greater variety of Lepidium cultivars with practical and attractive ornamental features. Additionally, a need exists for additional Lepidium virginicum cultivars that can be easily propagated by seed, with consistent results. The new Lepidium ‘DANLEPGRZR’ was developed through a controlled breeding program and exhibits unique, desirable and stable characteristics.

### SUMMARY OF THE INVENTION

The present invention provides Lepidium plant selections that produce a high quantity of lateral branches, resulting in many harvestable stems for cut flower purposes. Flowering stems of the new variety can be used in flower bouquets as filler plant material. The large quantity of stems produced by the new variety make this invention especially useful for commercial cut flower purposes. These qualities distinguish the new cultivar from typical Lepidium virginicum varieties.

These and other objectives have been achieved in accordance with the present invention which provides ‘DANLEPGRZR’ as a new Lepidium cultivar that is a product of a planned breeding program conducted by the inventor, Gavriel Danziger, in Moshav Mishmar Hashiva, Israel in 2018. The parent is the Lepidium virginicum inbred line identified by code LEV-17-9 (unpatented).

The parental cultivar has a sufficient degree of homozygosity such that the progeny of the cross are genetypically and phenotypically uniform. The new Lepidium virginicum ‘DANLEPGRZR’ therefore can be produced by sexual reproduction by crossing the parent inbred line identified by the code LEV-17-9 to produce a population of progeny plants, each of which has the combination of characteristics as herein disclosed for the new Lepidium virginicum ‘DANLEPGRZR’.

Seeds which are variety ‘DANLEPGRZR’ are produced by crossing the parental inbred line identified by the code LEV-17-9 and are to be deposited with the National Collection of Industrial Food and Marine Bacteria(NCIMB), Ferguson Building, Bucksburn, Aberdeen, Scotland.

### OBJECTS OF THE INVENTION

The present invention relates to seeds which produce Lepidium virginicum ‘DANLEPGRZR’. The present invention also relates to Lepidium plants, and parts thereof; having all the physiological and morphological characteristics of Lepidium virginicum ‘DANLEPGRZR’. The present invention relates to a plant produced from seeds which arc Lepidium virginicum ‘DANLEPGRZR’. The present invention also relates to plant parts, such as pollen, seeds or inflorescence produced by Lepidium virginicum ‘DANLEPGRZR’.

The present invention relates to a method of producing seed which are Lepidium virginicum ‘DANLEPGRZR’, by performing a self-crossing of Lepidium virginicum inbred line identified by code LEV-17-9 (unpatented) and harvesting seeds produced from said cross.

The present invention also relates to a method of producing plants having all the physiological and morphological characteristics of the Lepidium virginicum ‘DANLEPGRZR’ comprising the steps of (a) self-crossing Lepidium virginicum inbred line identified by code LEV-17-9 (unpatented) (b) harvesting seeds produced from said cross; and (c) producing plants from said harvested seeds.

The present invention also relates to producing progeny plants from the cross of Lepidium virginicum ‘DANLEPGRZR’, as the female or male parent, with another Lepidium plant, and selecting progeny plants from this cross.

### BRIEF DESCRIPTION OF THE PHOTOGRAPHS

The patent or application file contains at least one drawing executed in color. Copies of this patent or patent application publication with color drawings will be provided by the Office upon request and payment of the necessary fees.

The accompanying photographs illustrate the overall appearance of the new Lepidium virginicum ‘DANLEPGRZR’ showing the colors as true as is reasonably possible with colored reproductions of this type. Colors in the photographs may differ slightly from the color values cited in the detailed botanical description which accurately describes the color of ‘DANLEPGRZR’.

FIG. 1 shows a side view perspective of a typical flowering stem of ‘DANLEPGRZR’, on the lant.

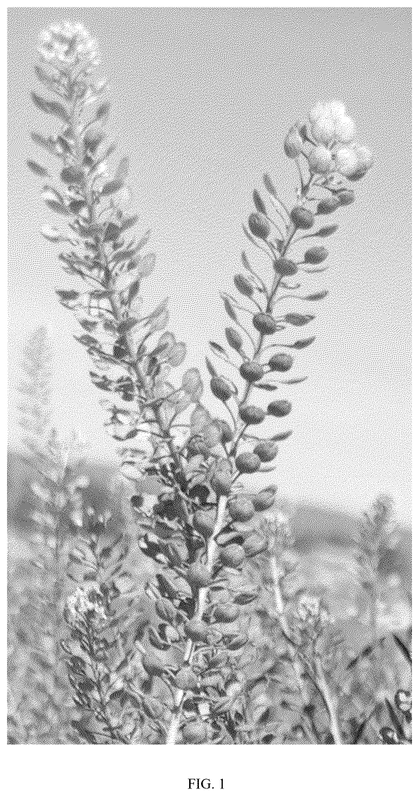

FIG. 2 shows a top view of a typical plant of ‘DANLEPGRZR’.

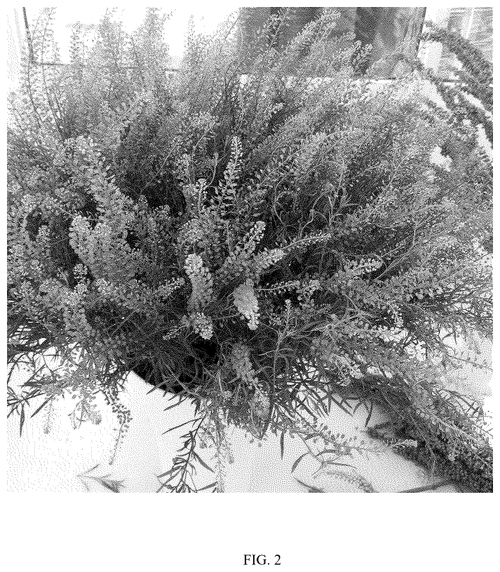

### DETAILED BOTANICAL DESCRIPTION

The present invention was created by the inventor, Gavriel Danziger during 2018, and flowered for the first time in 2019 in Mishmar Hashiva, Israel.

This invention is directed to Lepidium plant having all the morphological and physiological characteristics of the variety ‘DANLEPGRZR’ produced from seeds which are the product of the self-cross of the Lepidium virginicum inbred line identified by code LEV-17-9 (unpatented). The parent has a sufficient degree of homozygosity such that the progeny of the cross were, and continue to be, phenotypically uniform. The new variety ‘DANLEPGRZR’ can therefore be produced by sexual reproduction by crossing of the inbred selection identified by the code LEV-17-9 to produce a population of progeny plants, each of which has the combination of characteristics herein disclosed for the new variety ‘DANLEPGRZR’.

The following traits have been repeatedly observed and are determined to be unique characteristics of ‘DANLEPGRZR’ which in combination distinguish this Lepidium as a new and distinct cultivar:

### 1. High Volume Production of Long Stems

2. Strong and sturdy stems.
3. Vernalization requirement to flower.

The commercial cultivar known to the present inventor to be the most similar in comparison to the new Lepidium virginicum ‘DANLEPGRZR’ is the Lepidium cultivar ‘DANLEPVERGI’, U.S. Pat. No. 10,123,496. Plants of the new variety ‘DANLEPGRZR’ differ from plants of ‘DANLEPVERGI’ in the following:
1. Plants of ‘DANLEPGRZR’ produce a larger rosette than plants of ‘DLEPVERGI’.
2. Plants of ‘DANLEPGRZR’ flower later than plants of ‘DLEPVERGI’.
3. Plants of ‘DANLEPGRZR’ are taller than plants of ‘DLEPVERGI’.

‘DANLEPGRZR’ has not been tested and observed under all possible environmental conditions. The phenotype of the new cultivar may vary with variations in environment such as temperature, light intensity, frequency of fertilization, composition of fertilizer, acetylene treatment, day length and humidity, without any change in the genotype of the plant.

For example, substantial differences in plant height and diameter, number of leaves, and branches can result depending on the growing conditions. Typically, these plants are produced outdoors, and variations in temperature and humidity can produce different results.

The aforementioned photographs, together with the following observations, measurements and values describe the new Lepidium ‘DANLEPGRZR’ as grown in a greenhouse in Mishmar Hashiva, Israel, during late winter to spring under conditions which closely approximate those generally used in commercial practice. Plants of ‘DANLEPGRZR’ were grown with day temperatures ranging from about 18 °C to 35 °C and night temperatures ranging from about 8 °C to 20 °C. No artificial lighting or photoperiodic treatments were conducted.

Color references are made to the Royal Horticultural Society Colour Chart (RHS), 2005 mini edition, except where general colors of ordinary significance are used. Color values were taken under daylight conditions in a greenhouse Mishmar Hashiva, Israel. The age of the plants of ‘DANLEPGRZR’ described is about 13 weeks from planting a seedling plant. The seedling plant is approximately 5 weeks old.
BOTANICAL CLASSIFICATION: Lepidium virginicum

### Parentage

Parent: Lepidium virginicum inbred line identified by code LEV-17-9 (unpatented)

### Plant

Growth Habit: Upright
Height: Approximately 90 to 140 cm in Spring.
Plant Spread: Approximately 50 to 70 cm.
Growth Rate: Approximately 12 to 16 in Spring to achieve mature flowering stage.
Branching Characteristics: Pseudomonopodial.
Length of Primary Lateral Branches: 55 to 95 cm
Diameter of Lateral Branches: 2 to 4 mm.
Quantity of Primary Lateral Branches: Ranges between to 7-9 per plant.
Characteristics of Primary Lateral Branches:
   Color: Near RHS Yellow-Green 146D
   Texture: Glabrous
   Strength: Strong, Firm.
Internode length: Varies along the stem. In the summer-approximately 2.5 cm for the lower part of the stem, and approximately 1 cm for the upper part of the stem.

### Foliage

Leaf: Most foliage is basal. Cauline leaves occur as well.
The data refers to the basal leaves
Arrangement: Rosette
Quantity: Approximately 25 to 35 per branch in Spring
Average Length: 21.5 cm
Average Width: 4.5 cm
Shape of blade: Lyrate
Apex: Lobed
Base: Attenuate
Margin: Serrate
Texture of top surface: Scabrous
Texture of bottom surface: Scabrous
Appearance: top surface: Matte
Appearance bottom surface: Matte
Aspect: Convex
Color:
   Young foliage upper side: RHS Green N137B
   Young foliage under side: RHS Yellow-Green 147B
   Mature foliage upper side: RHS Green N137B
   Mature foliage under side: RHS Yellow-Green 147B
Venation:
   Type: Arcuate
   Venation color upper side: RHS Green 139D
   Venation color under side: RHS Yellow-Green 147B
Petiole:
   Length: 6 cm
   Diameter: 2 mm
   Color: RHS Green N137B
   Texture: Scabrous

### Inflorescence

Natural flowering season: Spring (February to May in Israel)
Days to flowering from a plantlet: Starting from seeds, approximately 2 weeks vernization followed by 6 to 8 weeks growth in Summer to the first flowering. From planting during Spring approximately 12 to 16 weeks.
Inflorescence and flower type and habit: The inflorescence type is a panicle and the flower type is single, cruciform, the habit is upright.
Rate of flower opening: 0.5 to 3 days from bud to fully opened flower.
Flower Longevity on Plant: 0.5 to 4 days.

### Persistent or Self-Cleaning: Self-Cleaning.

Vase life: Approximately 6 days.

### Bud:

Shape: Globose
Length: Average 1.5 mm
Diameter: Average 1.5 mm
Color: Near RHS Yellow-Green 144B

### Flower Size:

Diameter: Approximately Spring 3 mm.
Length: Approximately Spring 2 mm.

### Corolla/Petals:

Arrangement: Cruciform
Length: 1 mm
Width: 0.5 mm
Quantity: 4
Texture: Smooth
Apex: Obtuse
Base: Cuneate
Shape: Oblanceolate
Margin: Entire
Aspect: Convex
Color: When opening:
   Upper surface: RHS White NN155D
   Lower surface: RHS White NN155D
   Fully opened:
   Upper surface: RHS White NN155D
   Lower surface: RHS White NN155D
   Aging:
   Upper surface: RHS White NN155D
   Lower surface: RHS White NN155D

### Calyx/Sepals:

Quantity per flower: 4
Shape: Ovate
Length: 1 mm
Width: 0.5 mm
Apex: Acute
Base: Attenuate
Margin: Entire
Texture: Smooth
Color: Upper Surface: RHS Yellow-Green 144A
   Lower Surface: RHS Yellow-Green 144A
Peduncle:
   Length: Average range 8 to 1.2 mm.
   Diameter: 0.5 to 1.5 mm.
   Color: Yellow-Green 144A
   Orientation: Upright
   Texture: Smooth
Pedicel:
   Length: Approximately 4 to 6 mm
   Diameter: Approximately 0.5 to 1 mm
   Color: Yellow-Green 144A
   Orientation: Horizontal
   Texture: Smooth
Fragrance: Not fragrant

### Reproductive Organs

Stamens:
   Number: 2
   Filament length: Approximately 1 mm.
Anthers:
   Shape: Orbicular
   Length: Approximately 0.1-0.2 mm.
   Color: RHS Yellow-White 151B
Pistil:
   Style:
   Number: 1
      Length: Approximately 1 mm.
      Color: Yellow-Green 144A
   Stigma:
      Shape: Two lobed
      Color: Near RHS White 155A
      Ovary Color: Near RHS Green 143B

Temperature tolerance: Tolerates a range from approximately 8 °C to 40 °C

### Seeds/Fruit

### Fruits

Shape: Heart-shaped. Apically winged.

### Length: Approximately 3 mm.

### Width: Approximately 3 mm

Texture: Smooth. No veins.

### Seeds

### Shape: Napiform

### Length: 2 mm

### Width: 1 mm.

DISEASE/PEST RESISTANCE AND SUSCEPTIBILITY: Neither resistance nor susceptibility to normal diseases and pests of Lepidium observed.

We claim:

1. A Lepidium plant named ‘DANLEPGRZR’, representative seed to be deposited at the National Collection of Industrial Food and Marine Bacteria(NCIMB), Ferguson Building, Bucksburn, Aberdeen, Scotland.

2. A Lepidium seed that produces the plant of claim 1.

3. A plant part obtained from the Lepidium plant of claim 1.

4. A method for producing a Lepidium progeny hybrid plant comprising the steps of
(a) crossing Lepidium ‘DANLEPGRZR’, produced from representative seed to be deposited with the (NCIMB), as a female or male parent with another Lepidium plant, and
(b) selecting progeny.

5. The method according to claim 4, wherein the second Lepidium plant is ‘DANLEPGRZR’.
